(19) United States
(12) Patent Application Publication
(10) Pub. No.: US 2010/0273188 A1
(43) Pub. Date: Oct. 28, 2010

(54) Serum Prolactin Binding Protein in Epithelial Carcinoma

(75) Inventors: Charles CLEVENGER, Elmhurst, IL (US); William Sweet, Mountain View, CA (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/714,317

(22) Filed: Feb. 26, 2010

Related U.S. Application Data
(63) Continuation of application No. 11/552,498, filed on Oct. 24, 2006, now abandoned.
(60) Provisional application No. 60/596,829, filed on Oct. 24, 2005.

(51) Int. Cl. G01N 33/68; C07K 16/28; C07K 16/00; C07K 16/26

(52) U.S. Cl. 435/7.8; 530/387.1; 530/388.1; 530/388.22; 530/388.24; 436/501; 435/7.9

(57) ABSTRACT

The present invention relates to antibodies that have specificity towards prolactin binding protein (PRLBP) that is either bound to a binding partner or unbound to a binding partner, as well as antibodies towards PRLBP regardless of the binding state of PRLBP. The present invention also provides methods of using these PRLBP-specific antibodies, such as method of diagnosing and monitoring the progression of diseases such as epithelial carcinomas, osteoporosis, infertility and cachexia.

### CROSS REFERENCE TO RELATED APPLICATIONS

This application claims priority to U.S. Provisional Application No. 60/596,829, filed Oct. 24, 2005, which is incorporated by reference in its entirety.

### STATEMENT REGARDING FEDERALLY SPONSORED RESEARCH OR DEVELOPMENT

Part of the work performed during development of this invention utilized U.S. Government funds awarded by National Institutes of Health, Grant No. R01CA69294. The U.S. Government has certain rights in this invention.

### BACKGROUND OF THE INVENTION

1. Field of the Invention

The present invention relates to antibodies that have specificity towards prolactin binding protein (PRLBP) that is either bound to a binding partner or unbound to a binding partner, as well as antibodies specific to PRLBP, regardless of its binding state. The present invention also provides methods of using these PRLBP-specific antibodies, such as method of diagnosing and monitoring the progression of diseases such as epithelial carcinomas, osteoporosis, infertility and cachexia.

2. Background of the Invention

The prolactin receptor (PRLr) is a member of the cytokine receptor family, and Prolactin (PRL)-dependent signaling occurs due to the ligand-induced homodimerization of PRLr. PRL in mammary function by stimulating proliferation and differentiation of mammary epithelial cells by through the PRL receptor, which is present on epithelial cells throughout the body. (Clevenger et al, Endocr Rev., 24(1):1-27 (2003)). PRL function is mediated by a variety of signaling cascades and can also be attributed to the wide variety of PRLr forms observed in nature (see Clevenger et al, J. Endocrinol. 157(2): 187-197 (1998)). A long form and several other isoforms of PRLr are expressed in human tissues (Clevenger et al, Am. J. Pathol. 146(3): 695-705 (1995)). The nucleotide sequence of intermediate isoform of PRLr (which was identified from the breast cancer cell line T47D) is identical to the long isoform of PRLr, except for a 573-base pair deletion occurring at a consensus splice site that results in a frameshift and truncated intracytoplasmic domain (Kline et al., J. Biol. Chem. 274(50): 35461-35468 (1999)). The long isoform of PRLr is N-glycosylated (Dorato et al., Endocrinology 131(4): 1734-1742 (1992)) and since the extracellular domain of the intermediate isoform is identical to that of the long isoform, the glycosylation patterns are assumed to be similar. The molecular mass of glycosylated PRLr (intermediate isoform) is around 50 kDa (Kline et al., J. Biol. Chem. 274(50): 35461-35468 (1999)).

Recently it has been shown that the extra-cellular domain (ECD) of prolactin receptor (PRLr-ECD) is excised and secreted in the serum. This PRLr-ECD fragment, also known as prolactin-binding protein (PRLBP), is unique in that it can exist as a free form (unbound to a binding partner) or a bound form (bound to a binding partner). And while increased serum prolactin levels are associated with breast cancer (Hankinson, et al., JNC1 91:629-634 (1999)), alterations in the normal range of serum PRLBP could also be associated with several disease states, including but not limited to epithelial carcinomas such as breast carcinoma, prostate carcinoma, ovarian carcinoma, uterine carcinoma, cervical carcinoma, testicular carcinoma and the like, as well as osteoporosis, infertility and cachexia.

The free form of PRLBP is available to bind to a binding partner, thus inhibiting the actions the bound binding partner which can be, but is not limited to prolactin and growth hormone; and the bound form of PRLBP may prevent renal clearance of the binding partner, thus increasing the clearance time of the binding partner. It thus becomes important to determine the precise balance of uPRLBP and bPRLBP in diagnosing or monitoring various disease states, or monitoring the efficacy of treatments. Currently, there are no tests available for measuring serum PRLBP levels or any form. Accordingly, there exists a need in the art for compositions and methods to determine levels of total PRLBP, uPRLBP and bPRLBP to more precisely diagnose, monitor and treat various abnormalities associated with aberrant levels of serum PRLBP.

### SUMMARY OF THE INVENTION

The present invention provides antibodies, or functional fragments thereof, that are specific towards prolactin binding protein in a particular binding state. Specifically, the invention provides antibodies that have specificity towards unbound prolactin binding protein (uPRLBP), antibodies that have specificity towards bound prolactin binding protein (bPRLBP) and antibodies that can recognize both bound and unbound prolactin binding protein.

The present invention also provides methods of detecting bound prolactin binding protein (bPRLBP) in a sample, with the methods comprising contacting the sample with an antibody, or functional fragment thereof, that is specific for bPRLBP and detecting the binding of the antibody, or functional fragment thereof, to the bPRLBP. Likewise, the present invention provides methods of detecting unbound prolactin binding protein (uPRLBP) in a sample, with the methods comprising contacting the sample with an antibody, or functional fragment thereof, that is specific for uPRLBP and detecting the binding of the antibody, or functional fragment thereof, to the uPRLBP.

The present invention also relates to methods of diagnosing or testing for epithelial carcinoma in a patient, where the methods comprise contacting a sample from the patient with an antibody, or functional fragment thereof, that is specific for bPRLBP and detecting the binding of the antibody, or functional fragment thereof, to the bPRLBP. Likewise, the present invention provides methods of diagnosing or testing for epithelial carcinoma in a patient, with the methods comprising contacting the sample with an antibody, or functional fragment thereof, that is specific for uPRLBP and detecting the binding of the antibody, or functional fragment thereof, to the uPRLBP.

The present invention also provides methods of monitoring the progression of epithelial carcinoma in a patient, and methods of monitoring efficacy of treatments of epithelial carcinoma. In one set of embodiments these monitoring methods comprise measuring the levels of bPRLBP in a sample from a patient for at least two time points and determining the differences in the levels of bPRLBP between the two time points, where a difference in levels of bPRLBP may be indicative of the progression of the disease or of the efficacy of a treatment thereof. Likewise, in another set of embodiments, these monitoring methods comprise measuring the levels of uPRLBP in a sample from a patient for at least two time points and determining the differences in the levels of uPRLBP between the two time points, where a difference in levels of uPRLBP may be indicative of the progression of the disease or of the efficacy of a treatment thereof.

The present invention also relates to methods of diagnosing or testing for infertility in a subject, where the methods comprise contacting a sample from the patient with an antibody, or functional fragment thereof, that is specific for bPRLBP and detecting the binding of the antibody, or functional fragment thereof, to the bPRLBP. Likewise, the present invention provides methods of diagnosing or testing for infertility in a subject, with the methods comprising contacting the sample with an antibody, or functional fragment thereof, that is specific for uPRLBP and detecting the binding of the antibody, or functional fragment thereof, to the uPRLBP.

The present invention also provides methods of monitoring the treatment of infertility in a patient. In one set of embodiments, these monitoring methods comprise comparing the levels of detected bPRLBP between at least two time points in a subject receiving treatment for infertility, where differences in the levels of bPRLBP may be indicative of the effectiveness of the infertility treatment. In another set of embodiments, these monitoring methods comprise comparing the levels of detected uPRLBP between at least two time points in a subject receiving treatment for infertility, where differences in the levels of uPRLBP may be indicative of the effectiveness of the infertility treatment.

The present invention also provides for methods of diagnosing or testing for osteoporosis in a patient where the methods comprise contacting a sample from the patient with an antibody, or functional fragment thereof, that is specific for bPRLBP and detecting the binding of the antibody, or functional fragment thereof, to the bPRLBP. Likewise, the present invention provides methods of diagnosing or testing for osteoporosis in a patient, with the methods comprising contacting the sample with an antibody, or functional fragment thereof, that is specific for uPRLBP and detecting the binding of the antibody, or functional fragment thereof, to the uPRLBP.

The present invention also provides methods of monitoring the progression of osteoporosis in a patient, and methods of monitoring efficacy of treatments of osteoporosis. In one set of embodiments these monitoring methods comprise measuring the levels of bPRLBP in a sample from a patient for at least two time points and determining the differences in the levels of bPRLBP between the two time points, where a difference in levels of bPRLBP may be indicative of the progression of the disease or of the efficacy of a treatment thereof. Likewise, in another set of embodiments, these monitoring methods comprise measuring the levels of uPRLBP in a sample from a patient for at least two time points and determining the differences in the levels of uPRLBP between the two time points, where a difference in levels of uPRLBP may be indicative of the progression of the disease or of the efficacy of a treatment thereof.

The present invention also provides for methods of diagnosing or testing for cachexia in a patient where the methods comprise contacting a sample from the patient with an antibody, or functional fragment thereof, that is specific for bPRLBP and detecting the binding of the antibody, or functional fragment thereof, to the bPRLBP. Likewise, the present invention provides methods of diagnosing or testing for cachexia in a patient, with the methods comprising contacting the sample with an antibody, or functional fragment thereof, that is specific for uPRLBP and detecting the binding of the antibody, or functional fragment thereof to the uPRLBP.

### BRIEF DESCRIPTION OF THE DRAWINGS

FIG. 1 depicts a standard curve of an ELISA assay, using the antibodies of the present invention, for detecting serum levels of total prolactin binding protein (PRLBP). The data was generated using the antibodies of the present invention, wherein the range of concentrations was 0 ng/ml to 80 ng/ml of PRLBP. Linear regression of the data resulted in an R² value of 0.9981.

FIG. 2 depicts a bar graph of ELISA data assay, using the antibodies of the present invention, for detecting serum levels of total prolactin binding protein (PRLBP) The linear regression depicted in FIG. 1 was used to determine unknown serum levels of total PRLBP. The data indicate that only 10% of “normal patients” had detectable serum levels of total PRLBP, whereas 50% breast cancer patients had detectable levels of total PRLBP. The preliminary data indicate that detecting serum levels of may be useful to stratify patient populations and/or to diagnose or confirm a diagnosis of a cancer patient.

### DETAILED DESCRIPTION OF THE INVENTION

The present invention provides antibodies that are specific towards prolactin binding protein (PRLBP) in a particular binding state. As used herein, the term antibody is used to mean immunoglobulin molecules and functional fragments thereof, regardless of the source or method of producing the fragment. As used herein, a “functional fragment” of an immunoglobulin is a portion of the immunoglobulin molecule that specifically binds to a binding target. Thus, the term “antibody” as used herein encompasses whole antibodies, such as antibodies with isotypes that include but are not limited to IgG, IgM, IgA, IgD, IgE and IgY. Whole antibodies may be monoclonal or polyclonal, and they may be humanized or chimeric. The term “monoclonal antibody” as used herein is not limited to antibodies produced through hybridoma technology. Rather the term “monoclonal antibody” refers to an antibody that is derived from a single clone, including any eukaryotic, prokaryotic, or phage clone, and not the method by which it is produced. The term “antibody” also encompasses functional fragments of immunoglobulins, including but not limited to Fab fragments, Fab′ fragments, F(ab′)₂ fragments and Fd fragments. “Antibody” also encompasses fragments of immunoglobulins that comprise at least a portion of a V_{L} and/or V_{H} domain, such as single chain antibodies, a single-chain Fv (scFv), disulfide-linked Fvs and the like.

The antibodies of the present invention may be monospecific, bispecific, trispecific or of even greater multispecificity. In addition the antibodies may be monovalent, bivalent, trivalent or of even greater multivalency. Furthermore, the antibodies of the invention may be from any animal origin including, but not limited to, birds and mammals. In specific embodiments, the antibodies are human, murine, rat, sheep, rabbit, goat, guinea pig, horse, or chicken. As used herein, “human” antibodies include antibodies having the amino acid sequence of a human immunoglobulin and include antibodies isolated from human immunoglobulin libraries or from animals transgenic for one or more human immunoglobulin in and that do not express endogenous immunoglobulins, as described in U.S. Pat. No. 5,939,598, which is herein incorporated by reference.

The antibodies of the present invention may be described or specified in terms of the epitope(s) or portion(s) of a polypeptide to which they recognize or specifically bind. Or the antibodies may be described based upon their ability to bind to specific conformations of the antigen, such as the conformation of the antigen, e.g., PRLBP, when the antigen itself is bound to another molecule, such as PRL.

Antibodies of the present invention may also be described or specified in terms of their cross-reactivity, as well as their binding affinity towards the antigen. Specific examples of binding affinities encompassed in the present invention include but are not limited to those with a dissociation constant (Kd) less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹²M, 10⁻¹²M, 5×10⁻¹³ M, 10⁻¹³M, 5×10⁻¹⁴M, 10⁻¹⁴ M, 5×10⁻¹⁵ M, or 10⁻¹⁵ M.

The antibodies of the invention also include derivatives that are modified, for example, by covalent attachment of any type of molecule to the antibody such that covalent attachment does not prevent, the antibody from generating an anti-idiotypic response. Examples of modifications to antibodies include but are not limited to, glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin and the like. Additionally, the derivative may contain one or more non-classical amino acids.

In one embodiment of the present invention, the antibodies are specific towards PRLBP that is bound to a binding partner (bPRLBP). In a more specific embodiment, antibodies with specificity towards bPRLBP do not possess detectable binding affinity towards PRLBP that is unbound with its binding partner (uPRLBP). As used herein, a “binding partner” is a compound or molecule that is specifically bound to PRLBP. Binding partners of PRLBP include, but are not limited to prolactin and growth hormone (GH). Accordingly, one specific embodiment of the present invention provides for antibodies that are specific for bPRLBP, where the bPRLBP is bound to either PRL or GH. More specifically, the antibodies are specific for bPRLBP that is bound to PRL. Furthermore, PRLBP is “bound” to a binding partner if the binding protein and its binding partner are associated together, and the invention is not limited by the type of bond between the binding protein and its binding partner Examples of binding between PRLBP and its binding partner include, but are not limited to, covalent binding, non-covalent binding, hydrogen bonding, Van der Waals forces and the like.

in another embodiment of the present invention, the antibodies are specific towards PRLBP that is unbound to a binding partner (uPRLBP). In a more specific embodiment, antibodies with specificity towards uPRLBP do not possess detectable binding affinity towards bPRLBP.

In another embodiment, the antibodies of the present invention are specific towards PRLBP that is unbound to a binding partner. Specifically, the invention provides antibodies that have specificity towards unbound prolactin binding protein (uPRLBP), antibodies that have specificity towards hound prolactin binding protein (bPRLBP) and antibodies that can recognize both bound and unbound prolactin binding protein.

The antibodies of the present invention may be generated by any suitable method known in the art. Polyclonal antibodies to PRLBP can be produced by various procedures well known in the art. For example, a PRLBP can be administered to various host animals including, but not limited to, rabbits, mice, rats, to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al, Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (both of which are incorporated by reference).

Accordingly, one embodiment of the present invention provides antibodies that are secreted by hybridomas 1A2B1, A2D6, 1A8E7, 1H6C11 and 4F3B3. The 1A2B1 hybridoma was deposited with the American Type Culture Collection (ATCC), Manassas, Va. USA on or about Oct. 18, 2006 and has been assigned ATCC accession number. The deposit was made under the terms of the Budapest Treaty on the international recognition of the deposit of microorganisms for purposes of patent procedure. The strain will be irrevocably and without restriction or condition released to the public upon the issuance of a patent. The deposit is provided as a convenience to those of skill in the art and is not an admission that a deposit is required for enablement of the inventions described herein. The sequences of the polynucleotides contained in the deposited material, as well as the amino acid sequence of the polypeptide encoded thereby, are controlling in the event of any conflict with any description of sequences herein.

Methods for producing and screening for specific antibodies using hybridoma technology are routine and well known in the art. In a non-limiting example, mice can be immunized with a fusion protein of PRLBP-GST. Once an immune response is detected, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones can then be assayed by methods known in the art for cells that secrete antibodies capable of binding a polypeptide of the invention. Ascites fluid, which generally contains high levels of antibodies, can be generated by immunizing mice with positive hybridoma clones.

The antibodies of the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library. Phage expressing an antigen binding domain that binds the antigen of interest can be selected or identified with the antigen of interest, such as using a labeled antigen or antigen bound or captured to a solid surface or bead. The phage used in these methods are typically filamentous phage including, but not limited to, fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57191-280 (1994); PCT application No. PCT/0B91101134; PCT publications WO 90/02809; WO 91/10737; WO 92/01047; WO 92/18619; WO 93/1236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108, all of which are incorporated by reference.

Antibody fragments which recognize specific epitopes, e.g., uPRLBP or bPRLBP, may be generated by known techniques. For example, Fab and F(ab′)₂ fragments of the invention May be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

Other methods, such as recombinant techniques, may be used to produce Fab, Fab′ and F(ab′)₂ fragments and are disclosed in PCT publication WO 92/22324; Mullinax et al., BioTechniques 12(6):864-869 (1992); and Sawai et al., AJRI 34:26-34 (1995); and Better et al., Science 240:1041-1043 (1988), which are herein incorporated by reference. After phage selection, for example, the antibody coding regions from the phage can be isolated and used to generate whole antibodies, including human antibodies, or any other desired antigen binding fragment, and expressed in any desired host, including mammalian cells, insect cells, plant cells, yeast, and bacteria.

Examples of techniques which can be used to produce other types of fragments, such as scFvs and include those described in U.S. Pat. Nos. 4,946,778 and 5,258,498; Huston et al., Methods in Enzymology 203:46-88 (1991); Shu et al., Proc. Nat'l Acad. Sci. (USA) 90:7995-7999 (1993); and Skerra et al, Science 24011038-1040 (1988), all of which are incorporated by reference. For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985), Oi et al., BioTechniques 4:214 (1986); Gillies et al., J. Immunol. Methods 125:191-202 (1989); U.S. Pat. Nos. 5,807,715; 4,816,567; and 4,816,397, all of which are herein incorporated by reference. Humanized antibodies are antibody molecules from non-human species antibody that binds the desired antigen having one or more complementarity determining regions (CFAs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See U.S. Pat. No, 5,585,089; Riechmann et al., Nature 332:323 (1988), both of which are herein incorporated by reference. Antibodies can be humanized using a variety of techniques known in the art including, for example, CDR-grafting (EP 239,400; PCT publication WO 91/09967; U.S. Pat. Nos, 5,225,539; 5,530,101; and 5,585,089), veneering or resurfacing (EP 592.106; EP 519,596; Padlan, Molecular Immunology 28(4/5):489-498 (1991); Studnicka et al, Protein Engineering 7(6):805-814 (1994); Roguska. et al., Proc. Nat'l Acad. Sci. 91:969-913 (1994)), and chain shuffling (U.S. Pat. No. 5,565,332), all of which are hereby incorporated by reference.

Completely human antibodies may be particularly desirable for therapeutic treatment or diagnosis of human patients. Human antibodies can be made by a variety of methods known in the art including phage display methods described above using antibody libraries derived from human immunoglobulin sequences. See also. U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated by reference.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, such as PRLBP. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995), which is hereby incorporated by reference. For a detailed discussion of this technology for producing human antibodies and human monoclonal antibodies and protocols for producing such antibodies, see, e.g. PCT publications WO 98/24893: WO 92/01047; WO 96/34096; WO 9633735 European Patent No, 0 598 877; U.S. Pat. Nos. 5,413,923; 5,625,126; 5,633,425; 5,569,825; 5,661,016; 5,545,806 5,814.318; 5,885,793; 5916,771 and 5,939,598, which are incorporated by reference.

Still another approach for generating human antibodies utilizes a technique referred to as guided selection. In guided selection, a selected non-human monoclonal antibody, e.g., a mouse antibody, is used to guide the selection of a completely human antibody recognizing the same epitope. (Jespers et al., Biotechnology 12:899-903 (1988), herein incorporated by reference).

The antibodies of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays and the like. Such assays are routine and well-known in the art (see Ausubel et al., eds, 1994, Current Protocols in Molecular Biology, Vol 1, John Wiley & Sons. Inc., New York, which is incorporated by reference).

Immunoprecipitation protocols generally comprise lysing a population (if cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4″ C, adding protein A and/or protein G sepharose beads to the cell lysate, incubating for about an hour or more at C, washing the beads in lysis buffer and resuspending the beads in SDS/sample buffer. The ability of the antibody of interest to immunoprecipitate a particular antigen can be assessed by, e.g., Western blot analysis. One of skill in the art would be knowledgeable as to the parameters that can be modified to increase the binding of the antibody to an antigen and decrease the background binding.

Western blot analysis generally comprises preparing protein samples, electrophoresis of the protein samples in a polyacrylamide gel (e.g., 8%-20% SOS-PAGE depending on the molecular weight of the antigen), transferring the protein sample from the polyacrylamide gel to a membrane such as nitrocellulose, PVDF or nylon, blocking the membrane in blocking solution e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate, e.g. horseradish peroxidase or alkaline phosphatase or radioactive molecule (e.g., 32P or 125I) diluted in blocking buffer, washing the membrane in wash buffer, and detecting, the presence of the antigen. One of skill in the art would be knowledgeable as to the parameters that can be modified to increase the signal detected and to reduce the background noise.

For the purposes of assessing immunospecificity, ELISAs comprise preparing antigen, e.g., PRLBP and coating the well of a 96 well microliter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a label; instead, a labeled second antibody (which recognizes the antibody of interest) may be added to the well. One of skill in the art would be knowledgeable as to the parameters that can be modified to increase the signal detected as well as other variations of ELISAs known in the art. For the purposes of assaying for the presence of a species of PRLBP in a sample, the antibodies of the present invention may or may not be coated to the well, i.e., a capture antibody. As used herein the term “capture antibody” is used mean an antibody that immobilizes the antigen by specifically binding to the antigen. Further, an antigen is “immobilized” if the antigen or the antigen-antibody complex is separated or is capable of being separated from the remainder of the sample. When the capture antibody is coated to a well or other surface, a detection antibody may be added following the addition of the antigen of interest to the wells. As used herein, a detection antibody is used to mean an antibody comprising a label. In a specific embodiment, the methods of the present invention comprise the use of a capturing antibody and a detection antibody to detect the antigen, the capturing antibody or the capturing antibody-antigen complex.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassay, whereby the antigen is incubated with antibody of interest conjugated to a labeled compound (e.g., ³H or ¹²⁵I) in the presence of increasing amounts of an unlabeled second antibody.

Accordingly, using the antibodies described herein, the present invention also provides methods of detecting bPRLBP, uPRLBP and/or total PRLBP in a sample, with the methods comprising contacting the sample with a capture antibody that is specific for bPRLBP, uPRLBP and/or total PRLBP, respectively, and detecting the binding of the capture antibody to bPRLBP, uPRLBP and/or total PRLBP. The capture antibody may be coated onto a cell culture surface or a 96-well plate, such as an ELISA plate, or the capture antibody may be bound to or coated on beads or columns, or any surface or environment capable of housing the capture antibody such that it is available to bind to the antigen of interest.

PRLBP, either bound or unbound, is a useful biomarker and measured levels in a subject can be used to assess a disease state or condition, or can be used to assess the risk of developing a disease state or condition, such as, but not limited to, epithelial cancer, osteoporosis, cachexia, infertility. Of course, the methods of detecting PRLBP can be combined with detecting other biomarkers that are also indicative of particular disease states. For example, the methods of the present invention can be combined with methods of detecting biomarkers such as, but not limited to, c-myb, Nek-3, PIAS, SIM), leptin osteopontin. IGF-I and IGF-II to name a few.

In a specific embodiment, monoclonal antibodies consist of several unique clones, and each clone uniquely recognizes bPRLBP, uPRLBP or total PRLBP. As such, the monoclonal antibody is used as the capturing antibody to determine bPRLBP, uPRLBP and/or total PRLBP in the serum, and a polyclonal antibody is used as the detecting antibody, where the polyclonal antibody is labeled. In another embodiment, a monoclonal antibody is used to bind a captured antigen, and a labeled secondary antibody is then used to detect the antigen-antibody complex. For the purposes of the present invention, a secondary antibody is an antibody that is not the primary antibody and includes, but is not limited to, a detection antibody. The primary antibody is the antibody that initially binds to and/or captures the antigen in a sample. A secondary antibody may bind to the primary antibody, the antigen of interest or the antigen-primary antibody complex. The methods of the present invention comprise the use of zero, one, two, three, four or more secondary antibodies. In yet another embodiment, a polyclonal antibody is used to hind the captured antigen, and a labeled secondary antibody is used to detect the antibody-antigen complex. In other words, the primary or secondary antibodies can be either monoclonal, polyclonal or a combination thereof. In addition the primary antibody could be absorbed on many surfaces such as beads or microarrays for an automated method. A PRLBP ELISA kit can quantitatively measure the serum level of bPRLBP, uPRLBP and/or total PRLBP. In a more specific embodiment, monoclonal antibody secreted by hybridoma 1A2B1 was used as the capturing antibody. Briefly, the initial characterization of 1A2B1 clone reveals that it strongly recognizes long and intermediate human PRL receptor and PRLBP and moderately reacts with delta S1 isoform via Western blotting. In the serum, the 1A2B1 antibody captures the total PRLBP. Polyclonal antibody raised against PRLBP is used as the detection antibody.

As used herein, a sample can be any environment that may be suspected of containing the antigen of interest. Thus, a sample includes, but is not limited to, a solution, a cell, a body fluid, a tissue or portion thereof, and an organ or portion thereof. Examples of animal cells include, but are not limited to, insect, avian, and mammalian such as, for example, bovine, equine, porcine, canine, feline, human and nonhuman primates. The scope of the invention should not be limited by the cell type assayed Examples of biological fluids to be assayed include, but are not limited to, blood, plasma, serum, urine, saliva, milk, seminal plasma, synovial interstitial fluid, cerebrospinal fluid, lymphatic fluids, bile and amniotic fluid. The scope of the methods of the present invention should not be limited by the type of body fluid assayed. The terms “subject” and “patient” are used interchangeably herein and are used to mean an animal, particularly a mammal, more particularly a human or nonhuman primate.

The samples may or may not have been removed from their native environment. Thus, the portion of sample assayed need not be separated or removed from the rest of the sample or from a subject that may contain the sample. For example, the blood of a subject may be assayed for bPRLBP, uPRLBP and/or total PRLBP, without removing any of the blood from the patient. Of course, the sample may also be removed from its native environment. For example, the sample may be a tissue section that can be used in immunohistochemistry (IHC) techniques, and the antibodies of the present invention may be used in standard WIC techniques, where the antibodies are brought into contact with the sample and the binding of the antibody to the antigen is detected using in standard immunihistochemistry techniques. Furthermore, the sample may be processed prior to being, assayed. For example, the sample may be diluted or concentrated; the sample may be purified and/or at least one compound, such as an internal standard, may be added to the sample. The sample may also be physically altered (e.g., centrifugation, affinity separation) or chemically altered (e.g., adding an acid, base or buffer, heating) prior to or in conjunction with the methods of the current invention. Processing also includes freezing and/or preserving the sample prior to assaying.

The invention is not limited by the method of detecting the binding of the capture antibody to the antigen, e.g., bPRLBP and uPRLBP. For example, the detection of binding may include, but is not limited to, using a second detection antibody that binds to the capture antibody-antigen complex, such as in a “sandwich ELISA,” using spectrophotometry, such as mass spectroscopy, and electrophoresis, such as Western Blotting. The use of subsequent detection antibodies to detect binding of the antibody to the antigen may include, but is not limited to, radioactive isotopes and enzymes, such as horse radish peroxidase or alkaline phosphatase, as has been described herein. Additionally, if the capture antibody, for example, is bound to a bead or particle, methods of detecting and measuring bound antigen may also include flow cytometry (FACS).

Detection may also be accomplished by use of a labeled primary or labeled secondary antibody. A label, as used herein, is intended to mean a chemical compound or ion that possesses or comes to possess or is capable of generating a detectable signal. The labels of the present invention may be conjugated to the primary antibody or secondary antibody, the antigen of interest or a surface onto which the label and/or antibody is attached. Examples of labels includes, but are not limited to, radiolabels, such as, for example, ³H and ³²P, that can be measured with radiation-counting devices; pigments, dyes or other chromogens that can be visually observed or measured with a spectrophotometer; spin labels that can be measured with a spin label analyzer; and fluorescent labels (fluorophores), where the output signal is generated by the excitation of a suitable molecular adduct and that can be visualized by excitation with light that is absorbed by the dye or can be measured with standard fluorometers or imaging systems. Additional examples of labels include, but are not limited to, a phosphorescent dye, a tandem dye and a particle. The label can be a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The term label also includes a “tag” or hapten that can bind selectively to a conjugated molecule such that the conjugated molecule, when added subsequently along with a substrate, is used to generate a detectable signal. For example, one can use biotin as a label and subsequently use an avidin or streptavidin conjugate of horseradish peroxidate (HRP) to bind to the biotin label, and then use a calorimetric substrate tetramethylbenzidine (TMB)) or a fluorogenic substrate such as Amplex Red reagent (Molecular Probes, Inc.) to detect the presence of HRP. Numerous labels are know by those of skill in the art and include, but are not limited to, particles, fluorophores, haptens, enzymes and their colorimetric, fluorogenic and chemiluminescent substrates and other labels that are described in RICHARD P. HAUGLAND, MOLECULAR PROBES HANDBOOK OF FLUORESCENT PROBES AND RESEARCH PRODUCTS (9 edition, CD-ROM. (September 2002), which is herein incorporated by reference.

A fluorophore of the present invention is any chemical moiety that exhibits an absorption maximum beyond 280 nm, and when covalently attached to a labeling reagent retains its spectral properties. Fluorophores of the present invention include, without limitation; a pyrene (including any of the corresponding derivative compounds disclosed in U.S. Pat. No. 5,132,432, incorporated by reference), an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine (including any corresponding compounds in U.S. Ser. Nos. 09/968,401 and 09/969,853, incorporated by reference), a carbocyanine (including any corresponding compounds in U.S. Ser. Nos. 09/557,275; 09/969,853 and 09/968,401; U.S. Pat. Nos. 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445; and publications WO 02/26891, WO 97/40104, WO 99/51702, WO 01/21624; EP 1 065 250 A 1, incorporated by reference), a carbostyryl, a porphyrin, a salicylate, an anthranilate, an azulene, a perylene, a pyridine, a quinoline, a borapolyazaindacene (including any corresponding corn pounds disclosed in U.S. Pat. Nos. 4,774,339; 5,187,288; 5,248,782; 5,274,113; and 5,433,896, incorporated by reference), a xanthene (including any corresponding compounds disclosed in U.S. Pat. Nos. 6,162,931; 6,130,101; 6,229,055; 6,339,392; 5,451,343 and U.S. Ser. No. 09/922,333, incorporated by reference), an oxazine (including any corresponding compounds disclosed in U.S. Pat. No. 4,714,763, incorporated by reference) or a benzoxazine, a carbazine (including any corresponding compounds disclosed in U.S. Pat. No. 4,810,636, incorporated by reference), a phenalenone, a coumarin (including an corresponding compounds disclosed in U.S. Pat. Nos. 5,696,157; 5,459,276; 5,501,980 and 5,830,912, incorporated by reference), a benzofuran (including an corresponding compounds disclosed in U.S. Pat. Nos. 4,603,209 and 4,849,352, incorporated by reference) and benzphenalenone (including any corresponding compounds disclosed in U.S. Pat. No. 4,812,409, incorporated by reference) and derivatives thereof. As used herein, oxazines include resorufins (including any corresponding compounds disclosed in 5,242,805, incorporated by reference), aminooxazinones, diaminooxazines, and their benzo-substituted analogs.

When the fluorophore is a xanthene, the fluorophore is optionally a fluorescein, a rhodol (including any corresponding compounds disclosed in U.S. Pat. Nos. 5,227,487 and 5,442,045, incorporated by reference), or a rhodamine (including any corresponding compounds in U.S. Pat. Nos. 5,798,276; 5,846,737; U.S. Ser. No. 09/129,015, incorporated by reference). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171, incorporated by reference). Alternatively, the fluorophore is a xanthene that is bound via a linkage that is a single covalent bond at the 9-position of the xanthene. Preferred xanthenes include derivatives of 3H-xanthen-6-ol-3-one attached at the 9-position, derivatives of 6-amino-3H-xanthen-3-one attached at the 9-position, or derivatives of 6-amino-3H-xanthen-3-imine attached at the 9-position.

Preferred fluorophores of the invention include xanthene (rhodol, rhodamine, fluorescein and derivatives thereof) coumarin, cyanine, pyrene, oxazine and borapolyazaindacene. Most preferred are sulfonated xanthenes, fluorinated xanthenes, sulfonated coumarins, fluorinated coumarins and sulfonated cyanines. The choice of the fluorophore attached to the labeling reagent will determine the absorption and fluorescence emission properties of the labeling reagent and immuno-labeled complex. Physical properties of a fluorophore label include spectral characteristics (absorption, emission and stokes shift), fluorescence intensity, lifetime, polarization and photo-bleaching rate all of which can be used to distinguish one fluorophore from another.

Typically the fluorophore contains one or more aromatic or heteroaromatic rings, that are optionally substituted one or more times by a variety of substituents, including without limitation, halogen, nitro, cyano, alkyl, perfluoroalkyl, alkoxy, alkenyl, alkynyl, cycloalkyl, arylalkyl, acyl, aryl or heteroaryl ring system, benzo, or other substituents typically present on fluorophores known in the art.

In one aspect of the invention, the fluorophore has an absorption maximum beyond 480 nm. In a particularly useful embodiment, the fluorophore absorbs at or near 488 nm to 514 nm (particularly suitable for excitation by the output of the argon-ion laser excitation source) or near 546 nm (particularly suitable for excitation by a mercury arc lamp).

Many of fluorophores can also function as chromophores and thus the described fluorophores are also preferred chromophores of the present invention.

In addition to fluorophores, enzymes also find use as labels. Enzymes are desirable labels because amplification of the detectable signal can be obtained resulting in increased assay sensitivity. The enzyme itself may not produce a detectable signal but is capable of generating a signal by, for example, converting a substrate to produce a detectable signal, such as a fluorescent, colorimetric or luminescent signal. Enzymes amplify the detectable signal because one enzyme on a labeling reagent can result in multiple substrates being converted to a detectable signal. This is advantageous where there is a low quantity of target present in the sample or a fluorophore does not exist that will give comparable or stronger signal than the enzyme. The enzyme substrate is selected to yield the preferred measurable product, e.g. colorimetric, fluorescent or chemiluminescence. Such substrates are extensively used in the art, many of which are described in the MOLECULAR PROBES HANDBOOK, supra.

In a specific embodiment, a colorimetric or fluorogenic substrate and enzyme combination uses oxidoreductases such as horseradish peroxidase and a substrate such as 3,3′-diaminobenzidine (DAB) and 3-amino-9-ethylcarbazole (AEC), which yield a distinguishing color (brown and red, respectively). Other colorimetric oxidoreductase substrates that yield detectable products include, but are not limited to: 2,2-azino-bis(3-ethylbenzothiazoline-6-sulfonic acid) (ABTS), o-phenylenediamine (OPD), 3,3′,5,5′-tetramothylbenzidino (TMB), o-dianisidine, 5-aminosalicylic acid, 4-chloro-1-naphthol. Fluorogenic substrates include, but are not limited to, homovanillic acid or 4-hydroxy-3-methoxyphenylacetic acid, reduced phenoxazines and reduced benzothiazines, including Amplex® Red reagent and its variants (U.S. Pat. No. 4,384,042) and reduced dihydroxanthenes, including dihydrofiuoresceins (U.S. Pat. No. 6,162,931, incorporated by reference) and dihydrorhodamines including dihydrorhodamine 123. Peroxidase substrates that are tyramides (U.S. Pat. Nos. 5,196,306; 5,583,001 and 5,731,158, incorporated by reference) represent a unique class of peroxidase substrates in that they can be intrinsically detectable before action of the enzyme but are “fixed in place” by the action of a peroxidase in the process described as tyramide signal amplification (TSA). These substrates are extensively utilized to label targets in samples that are cells, tissues or arrays for their subsequent detection by microscopy, flow cytometry, optical scanning and fluorometry.

Another preferred colorimetric and in some cases fluorogenic) substrate and enzyme combination uses a phosphatase enzyme such as an acid phosphatase, an alkaline phosphatase or a recombinant version of such a phosphatase in combination with a colorimetric substrate such as 5-bromo-6-chloro-3-indolyl phosphate (SUP), 6-chloro-3-indolyl phosphate, 5-bromo-6-chloro-3-indolyl phosphate, p-nitrophenyl phosphate, or o-nitrophenyl phosphate or with a fluorogenic substrate such as 4-methylumbelliferyl phosphate, 6,8-difluoro-7-hydroxy-4-methylcoumarinyl phosphate (DiFMUP, U.S. Pat. No. 5,830,912, incorporated by reference) fluorescein diphosphate, 3-O-methylfluorescein phosphate, resorufin phosphate, 9H-(1,3-dichloro-9,9-dimethylacridin-2-one-7-yl) phosphate (DDAO phosphate), or ELF 97, ELF 39 or related phosphates (U.S. Pat. Nos. 5,316,906 and 5,443,986, incorporated by reference).

Glycosidases, in particular beta-galactosidase, beta-glucuronidase and beta-glucosidase, are additional suitable enzymes. Appropriate colorimetric substrates include, but are not limited to, 5-bromo-4-chloro-3-indolyl beta-D-galactopyranoside (X-gal) and similar indolyl galactosides, glucosides, and glucuronides, o-nitrophenyl beta-D-galactopyranoside (ON PG) and p-nitrophenyl beta-D-galactopyranoside. Preferred fluorogenic substrates include resorufin beta-D-galactopyranoside, fluorescein digalactoside (FDG), fluorescein diglucuronide and their structural variants (U.S. Pat. Nos. 5,208,148, 5,242,805; 5,362,628, 5,575,424 and 5,773,235, incorporated by reference), 4-methylumbelliferyl beta-D-galactopyranoside, carboxyumbelliferyl beta-D-galactopyranoside and fluorinated coumarin beta-D-galactopyranosides (U.S. Pat. No. 5,830,912, incorporated by reference).

Additional enzymes include, but are not limited to, hydrolases such as cholinesterases and peptidases, oxidases such as glucose oxidase and cytochrome oxidases, and reductases for which suitable substrates are known.

Specific embodiments of the present invention comprise enzymes and their appropriate substrates to produce a chemiluminescent signal, such as, but not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally useful.

Additional embodiments comprise haptens such as biotin. Biotin is useful because it can function in an enzyme system to further amplify the detectable signal, and it can function as a tag to be used in affinity chromatography for isolation purposes. For detection purposes, an enzyme conjugate that has affinity for biotin is used, such as avidin-HRP. Subsequently a peroxidase substrate is added to produce a detectable signal.

Haptens also include hormones, naturally occurring and synthetic drugs, pollutants, allergens, affector molecules, growth factors, chemokines, cytokines, lymphokines, amino acids, peptides, chemical intermediates, nucleotides and the like.

Fluorescent proteins also find use as labels for the labeling reagents of the present invention. Examples of fluorescent proteins include green fluorescent protein (GFP) and the phycobiliproteins and the derivatives thereof. The fluorescent proteins, especially phycobiliprotein, are particularly useful for creating tandem dye labeled labeling reagents. These tandem dyes comprise a fluorescent protein and a fluorophore for the purposes of obtaining a larger stokes shift wherein the emission spectra is farther shifted from the wavelength of the fluorescent protein's absorption spectra. This is particularly advantageous for detecting a low quantity of a target in a sample wherein the emitted fluorescent light is maximally optimized, in other words little to none of the emitted light is reabsorbed by the fluorescent protein. For this to work, the fluorescent protein and fluorophore function as an energy transfer pair wherein the fluorescent protein emits at the wavelength that the fluorophore absorbs at and the fluorophore then emits at a wavelength farther from the fluorescent proteins than could have been obtained with only the fluorescent protein. A particularly useful combination is the phycobiliproteins disclosed in U.S. Pat. Nos. 4,520,110; 4,859,582; 5,055,556, incorporated by reference, and the sulforhodamine fluorophores disclosed in U.S. Pat. No. 5,798,276, or the sulfonated cyanine fluorophores disclosed in U.S. Ser. Nos. 09/968/401 and 09/969/853, incorporated by reference; or the sulfonated xanthene derivatives disclosed in U.S. Pat. No. 6,130,101, incorporated by reference and those combinations disclosed in U.S. Pat. No. 4,542,104, incorporated by reference. Alternatively, the fluorophore functions as the energy donor and the fluorescent protein is the energy acceptor.

In one embodiment, the label is a fluorophore selected from the group consisting of fluorescein, coumarins, rhodamines, 5-TMRIA (tetramethylrhodamine-5-iodoacetamide), (9-(2(or 4)-(N-(2-maleimdylethyl)-sulfonamidyl)-4(or 2)-sulfophenyl)-2,3,6,7,12,13,16,17-octahydro-(1H,5H,11H,15H-xantheno(2,3,4-ij:5,6,7-i′j′)diquinolizin-18-ium salt) (Texas Red®), 2-(5-(1-(6-(N-(2-maleimdylethyl)-amino)-6-oxohexyl)-1,3-dihydro-3,3-dimethyl-5-sulfo-2H-indol-2-ylidene)-1,3-propyldienyl)-1-ethyl-3,3-dimethyl-5-sulfo-3,1-indolium salt (Cy™3), N,N′-dimethyl-N-(iodoacetyl)-N-(7-nitrobenz-2-oxa-1,3-diazol-4-yl)ethylenediamine (IANBD amide), 6-acryloyl-2-dimethylaminonaphthalene (acrylodan), pyrene, 6-amino-2,3-dihydro-2-(2-((iodoacetyl)amino)ethyl)-1,3-dioxo-1H-benz(de)isoquinoline-5,8-disulfonic acid salt (lucifer yellow), 2-(5-(1-(6-(N-(2-maleidylethyl)-amino)-6-oxohexyl)-1,3-dihydro-3,3-dimethyl-5-sulfo-2H-indol-2-ylidene)-1,3-pentadienyl)-1-ethyl-3,3-dimethyl-5-sulfo-3H-indolium salt (Cy™5), 4-(5-(4-dimethylaminophenyl)oxazol-2-yl)phenyl-N-(2-bromoacetamidoethyl)sulfonamide (Dapoxyl® (2-bromoacetamidoethyl)sulfonamide)), (N-(4,4-difluoro-1,3,5,7-tetramethyl-4-bora-3a,4a-diaza-s-indacene-2-yl)iodoacetamide (BODIPY® 507/545 IA), N-(4,4-difluoro-5,7-diphenyl-4-bora-3a,4a-diaza-s-indacene-3-propionyl)-N′-iodoacetylethylenediamine (BODIPY 530/550 IA), 5-((((2-iodoacetyl)amino)ethyl)amino)naphthalene-1-sulfonic acid (1,5-IAEDANS), and carboxy-X-rhodamine, 5/6-iodoacetamide (XRIA 5,6). Another example of a label is BODIPY-FL-hydrazide. Other luminescent labels include lanthanides such as europium (Eu3+) and terbium (Tb3+), as well as metal-ligand complexes of ruthenium [Ru(II)], rhenium [Re(I)], or osmium [Os(II)], typically in complexes with diimine ligands such as phenanthroline.

The present invention also relates to methods of diagnosing or testing for epithelial carcinoma in a patient. As used herein the term “diagnose” means to confirm the results of other tests or to simply confirm suspicions that the patient may have a particular disease or may have particular condition, such as infertility. A “test,” on the other hand, is used to indicate a screening method where the patient or the healthcare provider has no indication that the patient may, in fact, have a particular disease or particular condition. The methods of testing herein ma be used for a definitive diagnosis, or the tests may be used to assess a patient's likelihood or probability of developing a disease or condition.

The methods of diagnosing or testing for epithelial carcinoma in a patient comprise contacting a sample from the patient with a capture antibody that is specific for bPRLBP, uPRLBP and/or total PRLBP and detecting the binding of the antibody to bPRLBP, uPRLBP and/or total PRLBP, respectively. Examples of epithelial carcinomas that may be detected or tested, using the methods of the current invention include, but are not limited to, breast carcinoma, prostate carcinoma, ovarian carcinoma, cervical carcinoma, uterine carcinoma and testicular carcinoma. To diagnose or test for epithelial carcinoma, the levels a bPRLBP, uPRLBP and/or PRLBP in a sample may be compared to levels of bPRLBP, uPRLBP and/or total PRLBP, respectively, in a subject where the absence of epithelial carcinoma has been confirmed, i.e. normal levels.

As used herein “normal levels” of total PRLBP, bPRLBP and/or uPRLBP may be a range of values and may depend upon such factors as age, sex, sexual activity, ethnicity, level of body fat, weight, state of pregnancy or post-pregnancy, menstrual cycle, geographical location, general health of the patient, alcohol or drug, consumption, caffeine or nicotine intake and circadian rhythms. The normal levels of bPRLBP, uPRLBP and/or total PRLBP may be obtained from the same or a different patient from which the sample is obtained and the normal levels may be obtained from a single point or a population data points comprising wither multiple samples from a single patient or at least one sample taken from multiple patients. A difference in the levels of detected bPRLBP, uPRLBP and/or total PRLBP, compared to normal levels bPRLBP, uPRLBP and/or total PRLBP may be indicative that the patient is either at risk of developing a disease state or condition or has developed a disease state or condition.

The difference between detected levels of bPRLBP, uPRLBP and/or total PRLBP and normal levels, respectively, may be relative or absolute quantity. Of course, the difference may be equal to zero, indicating that the patient is normal, or that there has been no change in levels of measured antigen since the previous assay. The difference may simply be, for example, a measured fluorescent value, radiometric value, densitometric value, mass value etc., without any additional measurements or manipulations. Alternatively, the difference may be expressed as a percentage or ratio of the measured value of the antigen to a measured value of another compound including, but not limited to, a standard. The difference may be negative, indicating a decrease in the amount of measured antigen over normal value or from a previous measurement, and the difference may be positive, indicating an increase in the amount of measured antigen over normal values or from a previous measurement. The difference may also be expressed as a difference or ratio of the antigen to itself, measured at a different point in time. The difference may also be determined using in an algorithm, wherein the raw data is manipulated.

Similarly, the present invention also provides methods of monitoring the progression of epithelial carcinoma in a patient, and methods of monitoring efficacy of treatments of epithelial carcinoma. In one set of embodiments these monitoring methods comprise measuring the levels of bPRLBP, uPRLBP and/or total PRLBP in a sample from a patient in at least two time points and determining the differences in the levels of bPRLBP, uPRLBP and/or total PRLBP, respectively, between the two time points. Differences in the levels of bPRLBP, uPRLBP and/or total PRLBP between the two time points may be indicative of the progression of the disease or of the efficacy of a treatment thereof. Techniques for determining differences have been described previously herein. Based upon the results obtained from the methods described herein, a healthcare provider can individualize treatments for epithelial carcinomas. Accordingly, the invention provides for methods of individualizing a cancer therapy in a patient in need thereof.

The present invention also relates to methods of diagnosing or testing for infertility in a subject. These methods of diagnosing or testing for infertility in a patient comprise contacting a sample from the patient with a capture antibody that is specific for bPRLBP, uPRLBP and/or total PRLBP and detecting the binding of the antibody to bPRLBP, uPRLBP and/or total PRLBP, respectively. To diagnose or test for infertility, the levels of bPRLBP, uPRLBP and/or total PRLBP in a sample may be compared to levels of bPRLBP, uPRLBP and/or total PRLBP, respectively, in a subject where the absence of infertility has been confirmed, i.e., normal levels.

Likewise, the present invention also provides methods of monitoring the treatment of infertility in a patient. The methods of monitoring treatment of infertility comprise comparing the levels of detected bPRLBP, uPRLBP and/or total PRLBP between at least two time points in a subject receiving treatment for infertility, where differences in the levels of bPRLBP, uPRLBP and/or total PRLBP may be indicative of the effectiveness of the infertility treatment. Based upon the results obtained from the methods described herein, a healthcare provider can individualize treatments for infertility. Accordingly, the invention provides for methods of individualizing an infertility treatment in a patient in need thereof.

The present invention also provides methods of monitoring the fertility of a patient during a menstrual cycle. These methods of monitoring fertility in a patient comprise comparing the levels of detected bPRLBP, uPRLBP and/or total PRLBP between at least two time points in a subject during her menstrual cycle. Changes in levels of detected bPRLBP, uPRLBP and/or total PRLBP between the two or more time points may indicative that the subject is or is not fertile at a particular time point.

The present invention also provides for methods of diagnosing or testing for osteoporosis in a patient, with the methods comprising contacting a sample from the patient with a capture antibody that is specific for bPRLBP, uPRLBP and/or total PRLBP and detecting the binding of the antibody to bPRLBP, uPRLBP and/or total PRLBP, respectively. To diagnose or test for osteoporosis, the levels of bPRLBP, uPRLBP and/or PRLBP in a sample may be compared to levels of bPRLBP, uPRLBP and/or total PRLBP, respectively, in a subject where the absence of osteoporosis has been confirmed, i.e., normal levels.

The present invention also provides methods of monitoring the progression of osteoporosis in a patient, and methods of monitoring efficacy of treatments of osteoporosis. In one set of embodiments these monitoring methods comprise measuring the levels of bPRLBP, uPRLBP and/or total PRLBP in a sample from a patient in at least two time points and determining the differences in the levels of bPRLBP, uPRLBP and/or total PRLBP, respectively, between the two time points. Differences in the levels of bPRLBP, uPRLBP and/or total PRLBP between the two time points may be indicative of the progression of the disease or of the efficacy of a treatment thereof. Techniques for determining differences have been described previously herein. Based upon the results obtained from the methods described herein, a healthcare provider can individualize treatments for osteoporosis. Accordingly, the invention provides for methods of individualizing an osteoporosis therapy in a patient in need thereof.

The present invention also provides for methods of diagnosing or testing for cachexia in a patient with the methods comprising contacting a sample from the patient with a capture antibody that is specific for bPRLBP, uPRLBP and/or total PRLBP and detecting the binding of the antibody to bPRLBP, uPRLBP and/or total PRLBP, respectively. To diagnose or test for cachexia, the levels of bPRLBP, uPRLBP and/or PRLBP in a sample may be compared to levels of bPRLBP, uPRLBP and/or total PRLBP, respectively, in a subject where the absence of osteoporosis has been confirmed, i.e., normal levels.

The present invention also provides methods of monitoring the progression of cachexia in a patient, and methods of monitoring efficacy of treatments of cachexia. In one set of embodiments these monitoring methods comprise measuring the levels of bPRLBP, uPRLBP and/or total PRLBP in a sample from a patient in at least two time points and determining the differences in the levels of bPRLBP, uPRLBP and/or total PRLBP, respectively, between the two time points. Differences in the levels of bPRLBP, uPRLBP and/or total PRLBP between the two time points may be indicative of the progression of the disease or of the efficacy of a treatment thereof. Techniques for determining differences have been described previously herein. Based upon the results obtained from the methods described herein, a healthcare provider can individualize treatments for cachexia. Accordingly, the invention provides for methods of individualizing a cachexia therapy in a patient in need thereof.

The present invention also provides for kits for performing the methods described herein. Kits of the invention may comprise one or more containers containing one or more reagents useful in the practice of the present invention. Kits of the invention may comprise containers containing one or more buffers or buffer salts useful for practicing the methods of the invention. A kit of the invention may comprise a container containing a substrate for an enzyme. For example, a kit of the invention may comprise one or more substrates useful for detecting the enzymatic activity, i.e., horse radish peroxidase, or alkaline phosphatase.

Kits of the invention may comprise a container containing a stock antigen of known concentration. A stock of known concentration may be used to construct a calibration curve, for example. The calibration curve could then be used to determine the amount of antigen in a sample.

Kits of the invention may comprise one or more computer programs that may be used in practicing the methods of the invention. For example, a computer program may be provided that calculates a concentration of bPRLBP, uPRLBP and/or total PRLBP in a sample from results of the detecting levels of antibody bound to the antigen of interest. Such a computer program may be compatible with commercially available equipment, for example, with commercially available microplate readers. When determining the concentration of antigen in a sample, various dilutions of a stock of standard of known concentration may be applied to different wells in a microplate. Programs of the invention may take the output from microplate reader, prepare a calibration curve from the optical density observed in the wells and compare this densitometric reading to the optical density readings in wells with unknown amounts of antigen to determine how much antigen is present in the sample.

in view of the foregoing, the invention relates to at least the following enumerated embodiments. (1.) A method of diagnosing or testing for epithelial carcinoma in a patient, said method comprising detecting levels of prolactin binding protein (PRLBP) in a sample from said patient. (2) The method of enumerated embodiment 1, wherein said epithelial carcinoma is selected from the group consisting of breast carcinoma, prostate carcinoma, ovarian carcinoma, cervical carcinoma, uterine carcinoma and testicular carcinoma. (3) The method of enumerated embodiment 2, wherein said sample is selected from the group consisting of mammary tissue, prostate tissue, ovarian tissue, uterine tissue, cervical tissue, testicular tissue, liver tissue, blood, serum, plasma, milk, seminal plasma, and urine.

(4) The method of enumerated embodiment 3, wherein said detecting comprises the use of an antibody specific for total PRLBP. (5) The method of enumerated embodiment 4, wherein said antibody is a monoclonal antibody. (6) The method of enumerated embodiment 5, wherein said monoclonal antibody is secreted by hybridoma 1A2B1. (7) The method of enumerated embodiment 6, wherein said detecting further comprises a radioimmunoassay, an enzyme immunoassay, a spectrophotometric assay and an electrophoresis assay.

(8) A method of monitoring the progression of epithelial carcinoma in a patient, said method comprising (a) detecting levels of prolactin binding protein (PRLBP) in a sample from said patient at a first time point, (b) detecting levels of PRLBP in a sample from said patient at a second time point; and (c) comparing the levels of PRLBP at said first and second time points to determine a difference in the levels of PRLBP, wherein said difference in the levels of said PRLBP is indicative of the progression of said epithelial carcinoma in said patient. (9) The method of enumerated embodiment 8, wherein said epithelial carcinoma is selected from the group consisting of breast carcinoma, prostate carcinoma ovarian carcinoma, cervical carcinoma, uterine carcinoma and testicular carcinoma. (10) The method of enumerated embodiment 9, wherein said sample is selected from the group consisting of mammary tissue, prostate tissue, ovarian tissue, uterine tissue, cervical tissue, testicular tissue, liver tissue, blood, serum, plasma, milk, seminal plasma, and urine. (11) The method of enumerated embodiment 10, wherein said detecting comprises the use of an antibody specific for total PRLBP.

(12) The method of enumerated embodiment 11, wherein said antibody is a monoclonal antibody. (13) The method of enumerated embodiment 12, wherein said monoclonal antibody is secreted by hybridoma 1A2B1. (14) The method of enumerated embodiment 13, wherein said detecting further comprises a radioimmunoassay, an enzyme immunoassay, a spectrophotometric assay and an electrophoresis assay.

(15) A method of monitoring the treatment of epithelial carcinoma in a patient, said method comprising (a) detecting levels of prolactin binding protein (PRLBP) in a sample from said patient receiving treatment for epithelial carcinoma at a first time point, (b) detecting levels of PRLBP in a sample from said patient receiving treatment for epithelial carcinoma at a second time point; and (c) comparing the levels of PRLBP at said first and second time points to determine a difference in the levels of PRLBP, wherein said difference in the levels of said PRLBP is indicative of the effectiveness of said treatment of said epithelial carcinoma. (16) The method of enumerated embodiment 15, wherein said epithelial carcinoma is selected from the group consisting of breast carcinoma, prostate carcinoma, ovarian carcinoma, cervical carcinoma, uterine carcinoma and testicular carcinoma. (17) The method of enumerated embodiment 16, wherein said sample is selected from the group consisting of mammary tissue, prostate tissue, ovarian tissue, uterine tissue, cervical tissue, testicular tissue, liver tissue, blood, serum, plasma, milk, seminal plasma, and urine. (18) The method of enumerated embodiment 17, wherein said detecting comprises the use of an antibody specific for total PRLBP.

(19) The method of enumerated embodiment 18, wherein said antibody is a monoclonal antibody. (20) The method of enumerated embodiment 19, wherein said monoclonal antibody is secreted by hybridoma 1A281. (21) The method of enumerated embodiment 20, wherein said detecting further comprises a radioimmunoassay, an enzyme immunoassay, a spectrophotometric assay and an electrophoresis assay.

(22) A method of diagnosing infertility in a subject comprising detecting levels of prolactin binding protein (PRLBP) in a sample from said patient. (23) The method of enumerated embodiment 22, wherein said sample is selected from the group consisting of mammary tissue, prostate tissue, ovarian tissue, uterine tissue, cervical tissue, testicular tissue, liver tissue, blood, serum, plasma, milk, seminal plasma, and urine. (24) The method of enumerated embodiment 23, wherein said detecting comprises the use of an antibody specific for total PRLBP. (25) The method of enumerated embodiment 24, wherein said antibody is a monoclonal antibody. (26) The method of enumerated embodiment 25, wherein said monoclonal antibody is secreted by hybridoma 1A2B1. (27) The method of enumerated embodiment 26, wherein said detecting further comprises a radioimmunoassay, an enzyme immunoassay, a spectrophotometric assay and an electrophoresis assay.

(28) A method of monitoring the treatment of infertility in a patient, said method comprising (a) detecting levels of prolactin binding protein (PRLBP) in a sample from said patient receiving treatment for infertility at a first time point, (b) detecting levels of PRLBP in a sample from said patient receiving treatment for infertility at a second time point; and (c) comparing the levels of PRLBP at said first and second time points to determine a difference in the levels of PRLBP, wherein mid difference in the levels of said PRLBP is indicative of the effectiveness of said treatment of said infertility. (29) The method of enumerated embodiment 28, wherein said sample is selected from the group consisting of mammary tissue, prostate tissue, ovarian tissue, uterine tissue, cervical tissue, testicular tissue, liver tissue, blood, serum, plasma, milk, seminal plasma, and urine. (30) The method of enumerated embodiment 29, wherein said detecting comprises the use of an antibody specific for total PRLBP.

(31) The method of enumerated embodiment 30, wherein said antibody is a monoclonal antibody. (32) The method of enumerated embodiment 31, wherein said monoclonal antibody is secreted by hybridoma 1A2B1. (33) The method of enumerated embodiment 32, wherein said detecting further comprises a radioimmunoassay, an enzyme immunoassay, a spectrophotometric assay and an electrophoresis assay.

(34) A method of diagnosing osteoporosis in a patient comprising detecting levels of prolactin binding protein (PRLBP) in a sample from said patient. (35) The method of enumerated embodiment 34, wherein said sample is selected from the group consisting of mammary tissue, prostate tissue, ovarian tissue, uterine tissue, cervical tissue, testicular tissue, liver tissue, blood, serum, plasma, milk, seminal plasma, and urine. (36) The method of enumerated embodiment 35, wherein said detecting comprises the use of an antibody specific for total PRLBP. (37) The method of enumerated embodiment 36, wherein said antibody is a monoclonal antibody.

(38) The method of enumerated embodiment 37, wherein said monoclonal antibody is secreted by hybridoma 1A2B1. (39) The method of enumerated embodiment 38, wherein said detecting further comprises a radioimmunoassay, an enzyme immunoassay, a spectrophotometric assay and an electrophoresis assay.

(40) A method of monitoring the progression of osteoporosis in a patient, said method comprising (a) detecting levels of prolactin binding protein (PRLBP) in a sample from said patient at a first time point, (h) detecting levels of PRLBP in a sample from said patient at a second time point, and (c) comparing the levels of PRLBP at said first and second time points to determine a difference in the levels of PRLBP, wherein said difference in the levels of said PRLBP is indicative of the progression of said osteoporosis in said patient. (41) The method of enumerated embodiment 40, wherein said sample is selected from the group consisting of mammary tissue, prostate tissue, ovarian tissue, uterine tissue, cervical tissue, testicular tissue, liver tissue, blood, serum, plasma, milk, seminal plasma, and urine. (42) The method of enumerated embodiment 41, wherein said detecting comprises the use of an antibody specific for total PRLBP.

(43) The method of enumerated embodiment 42, wherein said antibody is a monoclonal antibody. (44) The method of enumerated embodiment 43, wherein said monoclonal antibody is secreted by hybridoma 1A2B1. (45) The method of enumerated embodiment 44, wherein said detecting further comprises a radioimmunoassay, an enzyme immunoassay, a spectrophotometric assay and an electrophoresis assay.

(46) A method of monitoring the treatment of osteoporosis in a patient, said method comprising (a) detecting levels of prolactin binding protein (PRLBP) in a sample from said patient receiving treatment for osteoporosis at a first time point, (b) detecting levels of PRLBP in a sample from said patient receiving treatment for osteoporosis carcinoma at a second time point, and (c) comparing the levels of PRLBP at said first and second time points to determine a difference in the levels of PRLBP, wherein said difference in the levels of said PRLBP is indicative of the effectiveness of said treatment of said osteoporosis.

(47) A method of diagnosing cachexia is a subject comprising detecting levels of prolactin binding protein (PRLBP) in a sample from said patient.

The Examples provided herein are illustrative and are not intended to limit the scope of the subject matter described herein.

### EXAMPLES

### Example 1

### Preparation and Selection of Antibodies Directed Towards uPRLBP and bPRLBP

### Example 1.1

### Production of Hybridomas

Three female BALB/c mice (Harlan Sprague-Dawley, Indianapolis, Ind., 20-week-old) were hyperimmunized against recombinantly produced human PRLBP-GST fusion protein. The mice were immunized (day 0) subcutaneously (0.1 ml) (1 mg/ml) with a preparation of antigen mixed with an equal volume of complete Freund's adjuvant. The mice were boosted with intraperitoneal injections of antigen preparation in 0.1 ml PBS (1 mg/ml) on days 21 and 42. On day 42, the mice were given an intravenous injection of antigen preparation in 0.1 ml PBS and the spleens were removed for fusion four days later. Spleen cells from the hyperimmunized mice were fused with P3-NS-1-Ag4-1 mouse myeloma cells as described by Kohler, G. et al, in Eur J. Immunol., 6:292-295 (1976), specifically incorporated herein by reference, in the presence of 50% polyethylene glycol (American Type Culture Collection, 1300-1600 MW) according to procedures established by Koprowski, H. et al, Proc. Natl. Acad. Sci. USA, 74:2985-2988 (1977), also incorporated herein by reference. After fusion, the cells were washed and resuspended in 300 ml of serum free medium containing 10×10⁻⁴ M hypoxanthine, 40×10⁻⁷ M aminopterin, 6.4×10⁻⁵M thymidine (HAT), and 50 gentamicin. The cells were then dispersed in 96-well microtiter plates in 0.2 ml aliquots. Hybridomas that were determined to be of interest, i.e., that produced monoclonal antibodies having binding specificity to PRLBP antigens but not to nonPRLBP antigens, were doubly cloned by limiting dilution. In this manner, 5 hybridomas producing monoclonal antibodies designated as 1A2B1, 1A2D6, 1A8E7, 1H6C11, and 4F3B3 were developed.

### Example 1.2

### Screening of Hybridoma Supernatants

To test for activity of monoclonal antibodies produced by the various hybridomas, 100 μl of GST-fusion protein antigen (1 μg/ml) was added to each well of a 96-well plate that was coated with rabbit anti-GST antibody. The plates were incubated at 37° C. for about 30 minutes and then rinsed four times with PBS-Tween buffer solution.

After washing, 100 μl of hybridoma cell culture supernatant was added to each well and the plate was incubated for about 30 minutes at 37° C. In addition, 100 μl of diluted specific mouse serum (1:1000) was used a positive control, and 100 μl of PBS, with 1% bovine serum albumin was used as a negative control.

After incubation, the plates were washed four times with PBS-Tween, and 100 μl of diluted HRP-Goat anti-mouse antibody (IgG+A+M (H+L)) (Invitrogen, Carlsbad, Calif. USA) (1:2000 dilution) or diluted HRP-Goat anti-mouse IgG (Invitrogen, Carlsbad, Calif. USA) was added to all wells of the plate and incubated at 37° C. for about 30 minutes. After incubation, the plates were rinsed four times with PBS-Tween and substrate solution (ABTS) was subsequently added to each well.

The colorimetric reaction was stopped after about 10 minutes with 1 M HCL and the plates read on an ELISA plate reader at 405/490 nm.

### Example 2

### Binding Assay Using Antibodies Specific for uPRLBP and bPRLBP

The monoclonal Ab produced by clone 1A2B1, which strongly recognizes both long and intermediate PRLr and moderately recognizes PRLBP, was used as the capture antibody. Briefly, 100 μl (100 μg/ml) of the “1A2B1 antibody” was applied to each well of an Immulon 2HB 96-well plate (ThermoLab Systems).

Recombinant fusion prolactin binding protein-GST was used to generate standard curves. Specifically, 100 μl of reconstituted PRL-BP GST was applied into wells labeled A1, A2 through F1, F2, to prepare a standard curve in duplicate. Normal human serum was added to wells G1 and G2; blank buffer solution (PBS) was added into wells H1 H2

After incubating for about 1 hour, the standards, serum and blank buffer solutions were removed and the wells washed 5 times with PBS-Tween solution to remove unbound PRLBP. After washing, anti-PRLBP rabbit polyclonal antibody conjugated to horse radish peroxidase (HRP) was used as a means of detection, and was applied to each well and incubated for about one hour. After this second incubation, the wells were washed 5 times with PBS-Tween to remove unbound detection antibody. After washing, HRP substrate (TMB) was applied to the wells and color was allowed to develop in darkness for about 15 minutes at room temperature. The color reaction was halted by the addition of 50 μl of 1M HCL to each well of the plate and the optical density of the wells was read on a plate reader at wavelengths of 450 nm and 595 nm.

The data were generated and analyzed using Molecular Devices/Emax precision microplate reader Pilot Run program and a standard curve of the formula below was generated.

y=0.014+1.68x−0.170x²

TABLE 1

| PRLr ECD Fusion | | |
|---|---|---|
| Protein, (μg/ml) | Mean, OD | Std. Dev |
| 1.280 | 1.886 | 0.129 |
| 0.320 | 0.534 | 0.014 |
| 0.080 | 0.148 | 0.011 |
| 0.020 | 0.048 | 0.004 |
| 0.005 | 0.022 | 0.001 |
| 0 | 0.000 | 0.000 |

Test serum samples were divided into three fractions and labeled as follows: (L) serum(non-processed), (L−) serum subjected to stripping such that is contains no reactive PRLBP, and (L+) serum subjected to non-stripping such that it contains reactive PRLBP, in addition, two types of controls were used: Zymed IgG sub-classes Isotyping Kit (Z) and PBS buffer (C). These controls are were processed similar to the serum: (Z) serum IgG (non-processed), (Z−) serum IgG subjected to stripping such that is contains no reactive PRLBP, and (Z+) serum subjected to non-stripping such that it contains reactive PRLBP and (C−) PBS subjected to stripping, and (C+) PBS subjected to non-stripping. The results of the binding assay are as follows

TABLE 2

| Sample | OD | μg/ml |
|---|---|---|
| L | 0.027 | 0.014 + 1.68 (0.027) − 0.170(0.027)² = 0.059 (59 ng) |
| L+ | 0.027 | 0.014 + 1.68 (0.027) − 0.170(0.027)² = 0.059 (59 ng) |
| L− | 0.016 | 0.014 + 1.68 (0.016) − 0.170(0.016)² = 0.041 (41 ng) |
| Z | 0.022 | 0.014 + 1.68 (0.022) − 0.170(0.022)² = 0.051 (51 ng) |
| Z+ | 0.035 | 0.014 + 1.68 (0.035) − 0.170(0.035)² = 0.073 (73 ng) |
| Z− | 0.015 | 0.014 + 1.68 (0.015) − 0.170(0.015)² = 0.039 (39 ng) |
| C+ | 0.014 | 0.014 + 1.68 (0.014) − 0.170(0.014)² = 0.038 (38 ng) |
| C− | 0.017 | 0.014 + 1.68 (0.017) − 0.170(0.017)² = 0.043 (43 ng) |

The results in the table above show that sera with stripping and yielded similar results to control (C+ and C−), indicating that stripping effectively eliminated reactive PRLBP. The average concentration of PRLBP in these samples was determined to be about 40.2 ng/ml. In this study, the L serum PRLBP was measured to be about 18.8 ng/ml (59-40.2).

### Example 3

### Binding Characteristics of Various Antibodies

Four hybridoma clones were generated and identified as clones 1A2B1, 1H6C11, 4F3B3 and 1A8E7.

To test if each of the clones were specific towards the same epitope, we ran a standard ELISA procedure as described herein, using antibody 1A2B1 as the capture antibody that was coated onto the bottom of the ELISA plates, and also using the PRLBP-GST fusion protein as the binding partner. For detection, however, we used the separate antibodies that had been conjugated to horse radish peroxidase, using standard conjugation chemistry.

In a first set of twelve of wells, 100 μl of HRP-PRLBP antibody (1A2B1) at a concentration of 0.5 μl/ml in PBS-Tween was applied to each well as the detection antibody. In a second set of twelve wells, 100 μl of HRP-PRLBP antibody (1H6C11) at a concentration of 0.5 μl/ml in PBS-Tween was applied to each well as the detection antibody. In a third set of wells, 100 μl of HRP-PRLBP antibody (4F3B3) at a concentration of 0.5 μl/ml in PBS-Tween was applied to each well as the detection antibody. In a fourth set 100 μl of HRP-PRLBP antibody (1A8E7) at a concentration of 0.5 μl/ml in PBS-Tween was applied to each well.

Incubation with the detection antibody and the colorimetric reaction proceeded as above, and the plates were read at 450 nm/595 nm. The results of the competitive assay are below. The results indicate that Abs 1H6C11, 4F3B3 and 1A8E7 recognize different epitopes, compared to the 1A2B1 Ab.

TABLE 3

| | 1 | 2 | 3 | 4 | 5 | 6 | 7 | 8 | 9 | 10 | 11 | 12 |
|---|---|---|---|---|---|---|---|---|---|---|---|---|
| A | 0.557 | 0.435 | 0.225 | 0.221 | 0.178 | 0.156 | 0.114 | 0.121 | 0.118 | 0.118 | 0.120 | 0.121 |
| B | 2.082 | 1.733 | 0.571 | 0.579 | 0.210 | 0.221 | 0.114 | 0.110 | 0.084 | 0.087 | 0.075 | 0.068 |
| C | 4.000 | 2.927 | 1.629 | 1.550 | 0.553 | 0.486 | 0.228 | 0.226 | 0.127 | 0.148 | 0.112 | 0.120 |
| D | 2.701 | 2.504 | 0.978 | 0.991 | 0.587 | 0.591 | 0.461 | 0.479 | 0.464 | 0.451 | 0.407 | 0.394 |

### Example 4

### Distinguishing Patient Populations by Measuring Using Total Levels of Plasma PRLBP

Plasma was collected from 10 normal patients and 4 patients diagnosed with some form of breast cancer. Lyophilized PRL-BP Standard was reconstituted with 220 μL distilled water to obtain concentrations of 0.5, 20, 80, 320, and 1280 ng/mL of PRLBP. In addition, a blank buffer control (PBS-Tween-20) and a blank human serum control were made. Plasma samples were diluted by a factor of 50 (1.50 dilution) in PBS. The PRLBP standards and two sets of controls, as well as the plasma samples were applied to a 96-well plate as shown below:

TABLE 4

| | 1 | 2 | 3 | 4 | 5 | 6 | 7 | 8 | 9 | 10 | 11 | 12 |
|---|---|---|---|---|---|---|---|---|---|---|---|---|
| A | 1280 ng | 1280 ng | S#1 | S#1 | S#9 | S#9 | S#17 | S#17 | S#25 | S#25 | S#33 | S#33 |
| B | 320 ng | 320 ng | S#2 | S#2 | S#10 | S#10 | S#18 | S#18 | S#26 | S#26 | S#34 | S#34 |
| C | 80 ng | 80 ng | S#3 | S#3 | S#11 | S#11 | S#19 | S#19 | S#27 | S#27 | S#35 | S#35 |
| D | 20 ng | 20 ng | S#4 | S#4 | S#12 | S#12 | S#20 | S#20 | S#28 | S#28 | S#36 | S#36 |
| E | 5 ng | 5 ng | S#5 | S#5 | S#13 | S#13 | S#21 | S#21 | S#29 | S#29 | S#37 | S#37 |
| F | 0 ng | 0 ng | S#6 | S#6 | S#14 | S#14 | S#22 | S#22 | S#30 | S#30 | S#38 | S#38 |
| G | Control | Control | S#7 | S#7 | S#15 | S#15 | S#23 | S#23 | S#31 | S#31 | S#39 | S#39 |
| H | Blank | Blank | S#8 | S#8 | S#16 | S#16 | S#24 | S#24 | S#32 | S#32 | S#40 | S#40 |

The 96-well plate comprised monoclonal antibody 1A2B1 attached to the bottom of the plate, as a capture antibody. As discussed herein, the 1A2B1 antibody recognized both total PRLBP (bound and unbound PRLBP).

After application of the samples, standards and controls, the plate was incubated at about 37° C. for about 1 hour. After incubation, each well of the plates was washed at least one time with about 250 μL of PBS-Tween-20 butler. After washing, 100 μL of diluted HRP-Rabbit anti-PRL-BP was applied to each well of the plate and the plate was incubated at about 37° C. for about 1 hour. After incubation with the detection antibody, each well of the plate was washed at least one time with about 250 μL PBS-Tween-20 buffer.

After washing, about 100 μL of Tetramethylbenzidine (TMB) substrate was applied to each well and the plate was incubated at room temperature for about 15 minutes. The plate was covered or placed in a dark room during incubated to prevent direct light reaching the plate. Next, about 50 μL of Stop Solution was to each well to halt the colorimetric reaction.

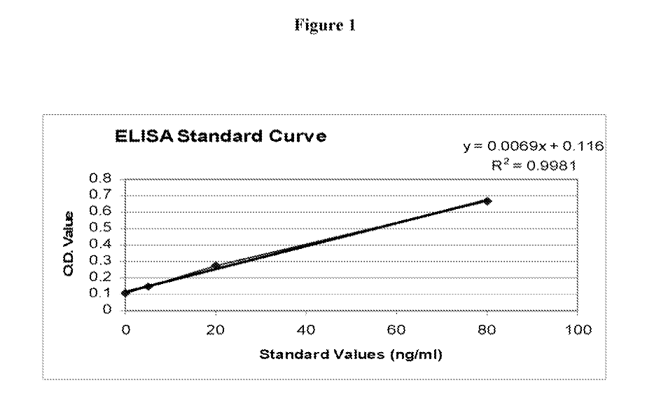

The plate was then read on a standard plate reader at A450 nm and A595 nm and the data was analyzed using absorbance at 450 nm. The plate reader software constructed a standard curve by plotting absorbance (y-axis) versus standard concentration (x-axis) to generate a line characterized by the formula y=0.0069x+0.116. The R² value, which is a measure of linearity of the standard curve, was 0.9981 (FIG. 1).

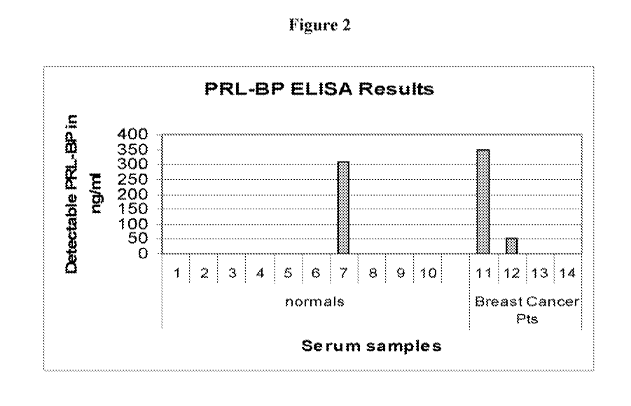

Using the above generated curve, the absorbance (y value) of each unknown was plugged into the equation and the equation was solved for x (concentration). Each value was then multiplied by the dilution factor (50), to determine the concentration of PRLBP in the serum of each patient tested. As can be seen in these preliminary studies, only 10% ( 1/10) of normal patients had detectable levels of serum PRLBP, whereas 50% ( 2/4) of the breast cancer patients had detectable levels of serum PRLBP (FIG. 2). The results indicate that the compositions and methods are useful in determining abnormal levels of serum PRLBP in patients. Furthermore, experimental results obtained using the compositions and methods of the present invention may be useful in stratifying or diagnosing patient populations of based upon serum PRLBP levels.

What is claimed is:

1. An antibody that has specificity towards prolactin binding protein that is both bound and unbound to a binding partner (total PRLBP).

2. The antibody of claim 2, wherein said antibody is a monoclonal antibody.

3. The antibody of claim 3, wherein said antibody is secreted by hybridoma 1A2B1.

4. An antibody that has specificity towards prolactin binding protein that is bound to a binding partner (bPRLBP).

5. The antibody of claim 4, wherein said antibody does not have specificity towards PRLBP that is unbound to said binding partner.

6. The antibody of claim 5, wherein said antibody is a monoclonal antibody.

7. The antibody of claim 6, wherein said binding partner is selected from the group consisting of prolactin and growth hormone.

8. The antibody of claim 7, wherein said binding partner is prolactin.

9. The antibody of claim 7, wherein said antibody is secreted by hybridoma 1A2D6 and 1A8E7.

10. An antibody that has specificity towards prolactin binding protein that is not bound to a binding partner (uPRLBP).

11. The antibody of claim 10, wherein said antibody does not have specificity towards PRLBP that is bound to said binding partner.

12. The antibody of claim 11, wherein said antibody is a monoclonal antibody.

13. The antibody of claim 12, wherein said antibody is secreted by hybridoma 1H6C11 and 4F3B3.

14. A method of detecting prolactin binding protein (PRLBP) in a sample, said method comprising
a) contacting, said sample with an antibody that is specific for said total PRLBP; and
b) detecting the binding of said antibody to said PRLBP.

15. The method of claim 14, wherein said sample is selected from the group consisting of mammary tissue, prostate tissue, testicular tissue, liver, blood, serum, plasma, milk, seminal plasma, and urine.

16. The method of claim 15, wherein said antibody is a monoclonal antibody.

17. The method of claim 16, wherein said monoclonal antibody is secreted by hybridoma 1 A2B1.

18. The method of claim 17, wherein said detecting comprises a radioimmunoassay, an enzyme immunoassay, a spectrophotometric assay and an electrophoresis assay.
